(19) United States
(12) Patent Application Publication
(10) Pub. No.: US 2025/0019730 A1
(43) Pub. Date: Jan. 16, 2025

(54) INTEGRATED PROCESS FOR THE PRODUCTION OF POLYHYDROXYALKANOATES AND BIOETHANOL FROM LIGNOCELLULOSE HYDROLYZATE

(71) Applicant: VERSALIS S.P.A., San Donato Milanese (MI) (IT)

(72) Inventors: Valentina RODIGHIERO, Novara (NO) (IT); Alessia ERCOLE, Rivalta Scrivia (AL) (IT); Daniele RIVA, Rivalta Scrivia (AL) (IT); Alessandro DEL SEPPIA, Mantova (MN) (IT); Tommaso PRANDO, Novara (NO) (IT); Alessandra FRATTINI, Rivalta Scrivia (AL) (IT)

(21) Appl. No.: 18/707,448

(22) PCT Filed: Nov. 2, 2022
(86) PCT No.: PCT/IB2022/060557

§ 371 Date: May 3, 2024

(30) Foreign Application Priority Data
Nov. 4, 2021 (IT) 102021000028109

(51) Int. Cl. C12P 7/625; C12P 7/10; C12P 7/14; C12R 1/05; C12R 1/07; C12R 1/185; C12R 1/38; C12R 1/865

(52) U.S. Cl. CPC C12P 7/625; C12P 7/10; C12P 7/14; C12P 2203/00; C12R 2001/05; C12R 2001/07; C12R 2001/185; C12R 2001/38; C12R 2001/865

(57) ABSTRACT

An integrated process for producing polyhydroxyalkanoates and bioethanol including:
(a) feeding a part of the lignocellulosic hydrolyzate to a first fermentation device in the presence of one microorganism capable of using sugars with six carbon atoms and organic acids, obtaining a first fermentation broth;
(b) subjecting the first broth to separation obtaining an aqueous suspension of cellular biomass having one polyhydroxyalkanoate and an aqueous phase having sugars with five carbon atoms in a quantity greater than or equal to 10 g/L;
(c) optionally, feeding a part of the aqueous phase from step (b), to a second fermentation device, obtaining a second fermentation broth (inoculum);
(d) feeding at least a part of the aqueous phase from step (b) and, optionally, the second broth and/or at least a part of lignocellulosic hydrolyzate, to a third fermentation device, obtaining a third fermentation broth; and
(e) subjecting the third broth to separation obtaining bioethanol.

### CROSS-REFERENCE TO RELATED APPLICATIONS

This application is a 35 U.S.C. § 371 National Stage patent application of PCT/IB2022/060557, filed on 2 Nov. 2022, which claims the benefit of Italian patent application 102021000028109, filed on 4 Nov. 2021, the disclosures of which are incorporated herein by reference in their entirety.

### TECHNICAL FIELD

The present disclosure relates to an integrated process for the production of polyhydroxyalkanoates (PHAs) and bioethanol from lignocellulosic hydrolyzate.

More in particular, the present disclosure relates to an integrated process for the production of polyhydroxyalkanoates (PHAs) and bioethanol from lignocellulosic hydrolyzate comprising the following steps: (a) feeding at least a part of said lignocellulosic hydrolyzate to a first fermentation device in the presence of at least one microorganism capable of using sugars with six carbon atoms (C6) and organic acids, obtaining a first fermentation broth; (b) subjecting the first fermentation broth obtained in said step (a) to separation obtaining an aqueous suspension of cellular biomass comprising at least one polyhydroxyalkanoate (PHA) and an aqueous phase comprising sugars with five carbon atoms (C5) in a quantity greater than or equal to 10 g/L, preferably between 12 g/L and 100 g/L; (c) optionally, feeding at least a part of the aqueous phase obtained in said step (b) to a second fermentation device in the presence of at least one microorganism capable of using both sugars with five carbon atoms (C5) and sugars with six carbon atoms (C6), obtaining a second fermentation broth (inoculum); (d) feeding at least a part of the aqueous phase obtained in said step (b) and, optionally, the second fermentation broth (inoculum) obtained in said step (c) and/or at least a part of said lignocellulosic hydrolyzate, to a third fermentation device in the presence of at least one microorganism capable of using both sugars with five carbon atoms (C5) and sugars with six carbon atoms (C6), obtaining a third fermentation broth; (e) subjecting said third fermentation broth to separation obtaining bioethanol.

The aforementioned polyhydroxyalkanoates (PHAs) can be advantageously used in various applications, in particular in the medical, pharmacological, agricultural, engineering and food fields. The aforementioned bioethanol can be advantageously used as it is, or mixed with fossil fuels, for automotive purposes, or, suitably purified, in the production of biochemicals (for example, disinfectants).

### BACKGROUND

The production of bioethanol and polyhydroxyalkanoates (PHAs) is known in the art.

In particular, amongst the biomasses of vegetable origin useful for the production of bioethanol we can list the crops of cereals such as, for example, corn, wheat, barley, or the crops of sugar cane, from which, through the fermentation of starches, it is possible to obtain bioethanol while, for the production of polyhydroxyalkanoates (PHAs), it is possible to list the crops of cereals such as, for example, corn, wheat, barley, from the starch of which sugars with 6 carbon atoms (C6) are obtained (for example, glucose), or the cultivation of vegetable species with a high content of triglycerides or oils (i.e. oleagineous species) such as, for example, rapeseed, soy, sunflower, palm.

For example, Nonato R. V. et al., in “Integrated production of biodegradable plastic, sugar and ethanol”, “Applied Microbiology and Biotechnology” (2001), Vol. 57, pg. 1-5, describe the use of secondary products deriving from the production of sugars and ethanol from biomass (i.e. sugar cane) as solvents for the extraction of poly-3-hydroxybutyric acid (PHB).

Recently, with the development of new technologies, lignocellulosic biomasses have been added to these biomasses of vegetable origin useful for the production of biofuels.

The use of lignocellulosic biomass in the production of biofuels is advantageous from many points of view, especially compared with the use of the crops of cereals and oleagineous species listed above, as said lignocellulosic biomasses are:
(i) widely available, for example, as waste products from the wood industry, the food industry, and also from the crops mentioned above;
(ii) inexpensive;
(iii) not in competition with products grown for human nutrition and, consequently, with the use of land cultivated for this purpose.

The use of these lignocellulosic biomasses that are already part of a biorefinery flow (not in competition with the food chain), represents a promising way for the sustainable production of polyhydroxyalkanoates (PHAs) and bioethanol.

Lignocellulosic biomass includes agricultural and forestry residues and, from a circular economy perspective, represents a category of materials with high potential for the industrial-scale production of a wide range of products from renewable sources. This is a category of materials generated as by-products of processes applied on large volumes such as those of the food and paper industry and for this reason they are generally available in abundance and at low costs.

The lignocellulosic material is mainly made up of cellulose, hemicellulose and lignin. Cellulose and hemicellulose are polysaccharides that can be hydrolyzed to simple sugars used as a substrate for the fermentation of microorganisms such as, for example, yeasts and bacteria. The process of hydrolysis, which leads to the breaking of polysaccharide bonds resulting in the formation of sugars with five carbon atoms (C5) (for example, xylose) and sugars with six carbon atoms (C6) (such as, for example, glucose), at the same time, leads to the production of inhibitory compounds which, in most cases, slow down the growth of microorganisms and reduce the production yields of the desired products.

The inhibitory compounds that are normally identified in lignocellulosic hydrolyzates are light carboxylic acids (for example, formic acid, acetic acid), compounds that result from the degradation of sugars [for example, furfural (F), 5-hydroxy-methyl-furfural (HMF)] and phenolic compounds.

For example, the presence of said light carboxylic acids can compromise the productivity of bioethanol up to a reduction of 67%-70% as reported, for example, by Lawford H. G. et al., in “Performance testing of Zymomonas mobilis metabolically engineered for cofermentation of glucose, xylose, and arabinose”, “Applied Biochemistry and Biotechnology” (2002), Vol. 98, pg. 429-448, who observed a reduction in bioethanol productivity in the gram-negative bacterium Zymomonas mobilis when the acetic acid concentration increased from 0 g/L a 2.5 g/L.

Casey E. et al., in “Effect of acetic acid and pH on the cofermentation of glucose and xylose to ethanol by a genetically engineered strain of Saccharomyces cerevisiae”, “FEMS Yeast Research” (2010), Vol. 10, pg. 385-393, describe the effect of acetic acid on the cofermentation of glucose and xylose operating under controlled pH conditions, by a genetically modified strain of Saccharomyces cerevisiae yeast. The presence of acetic acid leads to a significant reduction in the concentration of cellular biomass and, in particular, the rate of consumption of glucose and xylose decreases as the concentration of acetic acid increases, with a greater inhibitory effect as regards xylose.

To eliminate or reduce the negative effect created by said inhibitory compounds, it may be convenient to place a detoxification step of the lignocellulosic hydrolysates before fermentation.

For this purpose, various approaches have been developed which involve the application of chemical, physical or biological methods to eliminate, at least in part, the concentration of said inhibitory compounds as described, for example, by Mussatto I. S. et al., in “Alternatives for detoxification of diluted-acid lignocellulosic hydrolyzates for use in fermentative processes: a review”, “Bioresource Technology” (2004), Vol. 93, pg. 1-10.

Chakraborty P. et al., in “Conversion of volatile fatty acids into polyhydroxyalkanoate by Ralstonia eutropha”, “Journal of Applied Microbiology” (2009), Vol. 106, pg. 1996-2005, describe the use of by-products deriving from the production of ethanol, in particular of volatile fatty acids such as, for example, acetic acid, butyric acid, propionic acid, lactic acid, more particularly butyric acid and propionic acid, at aim to produce a product with high added value, in particular polyhydroxyalkanoate (PHA).

Schneider H. in “Selective removal of acetic acid from hardwood-spent sulfite liquor using a mutant yeast”, “Enzyme and Microbial Technology” (1996), Vol. 19, pg. 94-98, describes a process that uses a species of recombinant Saccharomyces cerevisiae yeast to selectively remove the acetic acid present in the lignocellulosic hydrolyzate. The mutant yeast is unable to metabolize sugars such as xylose, glucose, mannose or fructose, which will later be used in fermentation and within 24 hours the acetic acid levels in the culture medium drop from 6.8 g/L to less of 0.4 g/L.

Lisbeth O. et al., in “Fermentation of lignocellulosic hydrolysates for ethanol production”, “Enzyme and Microbial Technology” (1996), Vol. 18, Issue 5, pg. 312-331, describe a biological method for the purification of hydrolysates which consists in adapting the microorganism to grow on higher concentrations of inhibitory compounds. Said method, which is based on successive fermentations, uses the microorganisms of each fermentation as an inoculum for the next.

International patent application WO 2009/017441 describes a biological detoxification process using a recombinant strain of Saccharomyces cerevisiae yeast.

American patent U.S. Pat. No. 7,067,303 describes a biological detoxification process which uses a Coniochaeta lignaria (teleomorph) fungus or its Lecythophora (anamorph) state.

European patent application EP 2308959 describes a recombinant microorganism, i.e., Cupriavidus Brasilensis HMF 14, capable of detoxifying the lignocellulosic hydrolyzate mainly using furfural and 5-hydroxymethylfurfural as the main carbon source without affecting the sugar content that can subsequently be used in fermentation.

However, the aforementioned detoxification processes may present some drawbacks such as, for example:
a pre-treatment with genetically modified microorganisms or fungi is necessary in order to remove the inhibitory compounds with an increase in costs and lengthening of process times;
the microorganisms used can adapt to grow on higher concentrations of organic acids but said organic acids are not converted into products with high added value such as, for example, polyhydroxyalkanoates (PHAs);
the microorganisms used as well as being capable to convert the inhibitory compounds, often also metabolize sugars, making them unavailable for subsequent fermentation after a valuable product, thus lowering the overall yields of the fermentation process.

Various biological purification processes are therefore known in the state of the art, but, so far, no complete and effective method in terms of cost and productivity has been developed to fully exploit the components that are formed following the hydrolysis process of the lignocellulosic biomass.

Also known are microorganisms capable of metabolizing the inhibitory compounds present in the lignocellulosic hydrolyzate such as, for example, formic acid, acetic acid, furfural and phenolic compounds as described, for example, in Jiang G. et al., in “Carbon Sources for Polyhydroxyalkanoates and an Integrated Biorefinery”, “International Journal of Molecular Sciences.” (2016), Vol. 17(7), 1157.

Yu J. et al., in “Microbial utilization and biopolyester synthesis of bagasse hydrolysates”, “Bioresource Technology” (2008), Vol. 99, pg. 8042-8048, describe the production of polyhydroxyalkanoates (PHAs) starting from bagasse hydrolyzate deriving from sugar cane using the microorganism Ralstonia eutropha. The major organic inhibitors such as formic acid, acetic acid, furfural and acid-soluble lignin, are effectively used and removed at a low concentration (less than 100 ppm) and at the same time polyhydroxyalkanoates (PHAs) are synthesised which are accumulated in quantities equal to 57% by weight of the cell mass in the presence of appropriate C/N ratios.

Dietrich K. et al., in “Sustainable PHA production in integrated lignocellulose biorefineries”, “New BIOTECHNOLOGY” (2019), pg. 161-168, describe processes which use sugars with five carbon atoms (C5) in order to obtain polyhydroxyalkanoates (PHAs) with much lower yields than the processes which use glucose.

### SUMMARY

The Applicant therefore posed the problem of finding an integrated process for the production of polyhydroxyalkanoates (PHAs) and bioethanol with high yields.

The Applicant has now found that the production of polyhydroxyalkanoates (PHAs) and bioethanol can be advantageously carried out by means of an integrated process for the production of polyhydroxyalkanoates (PHAs) and bioethanol from lignocellulosic hydrolyzate comprising the following steps: (a) feeding at least a part of said lignocellulosic hydrolyzate to a first fermentation device in the presence of at least one microorganism capable of using sugars with six carbon atoms (C6) and organic acids, obtaining a first fermentation broth; (b) subjecting the first fermentation broth obtained in said step (a) to separation obtaining an aqueous suspension of cellular biomass comprising at least one polyhydroxyalkanoate (PHA) and an aqueous phase comprising sugars with five carbon atoms (C5) in a quantity greater than or equal to 10 g/L, preferably between 12 g/L and 100 g/L; (c) optionally, feeding at least a part of the aqueous phase obtained in said step (b) to a second fermentation device in the presence of at least one microorganism capable of using both sugars with five carbon atoms (C5) and sugars with six carbon atoms (C6), obtaining a second fermentation broth (inoculum); (d) feeding at least a part of the aqueous phase obtained in said step (b) and, optionally, the second fermentation broth (inoculum) obtained in said step (c) and/or at least a part of said lignocellulosic hydrolyzate, to a third fermentation device in the presence of at least one microorganism capable of using both sugars with five carbon atoms (C5) and sugars with six carbon atoms (C6), obtaining a third fermentation broth; (e) subjecting said third fermentation broth to separation obtaining bioethanol.

Numerous are the advantages obtained by means of this process. For example:
possibility of carrying out the integrated production of polyhydroxyalkanoates (PHAs) and bioethanol in the face of an effective use of carbon sources;
full use of residual sugars coming out of the production process of polyhydroxyalkanoates (PHAs) starting from lignocellulosic hydrolyzates thanks to integration with the bioethanol production process;
use of the organic acids contained in the lignocellulosic hydrolysates in the production of polyhydroxyalkanoates (PHAs) thus sending an aqueous phase with a reduced content of inhibitory compounds to the bioethanol production process.

Furthermore, this process allows to:
improve environmental sustainability relating to the production of polyhydroxyalkanoates (PHAs) on an industrial scale as lignocellulosic hydrolyzates do not compete with the human food chain;
reduce the production costs of both polyhydroxyalkanoates (PHAs) and bioethanol, thanks to the efficient and complete consumption of the sugars and acids contained in the lignocellulosic hydrolyzate and to the increase in the overall yield of the process of converting carbon sources into polyhydroxyalkanoates (PHAs) and bioethanol.

Therefore, the present disclosure provides an integrated process for the production of polyhydroxyalkanoates (PHAs) and bioethanol from lignocellulosic hydrolyzate comprising the following steps:
(a) feeding at least a part of said lignocellulosic hydrolyzate to a first fermentation device in the presence of at least one microorganism capable of using sugars with six carbon atoms (C6) and organic acids, obtaining a first fermentation broth;
(b) subjecting the first fermentation broth obtained in said step (a) to separation obtaining an aqueous suspension of cellular biomass comprising at least one polyhydroxyalkanoate (PHA) and an aqueous phase comprising sugars with five carbon atoms (C5) in a quantity greater than or equal to 10 g/L, preferably between 12 g/L and 100 g/L;
(c) optionally, feeding at least a part of the aqueous phase obtained in said step (b) to a second fermentation device in the presence of at least one microorganism capable of using both sugars with five carbon atoms (C5) and sugars with six carbon atoms (C6), obtaining a second fermentation broth (inoculum);
(d) feeding at least a part of the aqueous phase obtained in said step (b) and, optionally, the second fermentation broth (inoculum) obtained in said step (c) and/or at least a part of said lignocellulosic hydrolyzate, to a third fermentation device in the presence of at least one microorganism capable of using both sugars with five carbon atoms (C5) and sugars with six carbon atoms (C6), obtaining a third fermentation broth;
(e) subjecting said third fermentation broth to separation obtaining bioethanol.

For the purpose of the present description and of the following claims, the definitions of the numerical ranges always include the extremes unless otherwise specified.

For purposes of the present description and of the following claims, the term “comprising” also includes the terms “which essentially consists of” or “which consists of”.

For the purpose of the present description and of the following claims, the term “sugars with 5 carbon atoms (C5)” means pentose sugars, or more simply pentoses, which are monosaccharide carbohydrates composed of five carbon atoms having the chemical formula C₅H₁₀O₅. Similarly, for the purpose of the present description and of the following claims, the term “sugars with 6 carbon atoms (C6)” means hexose sugars, or more simply hexose, which are monosaccharide carbohydrates composed of six carbon atoms having a chemical formula C₆H₁₂O₆.

Polyhydroxyalkanoates (PHAs) are fully biodegradable polymers of microbial origin synthesized by prokaryotic microorganisms as an energy reserve and, in most cases, are accumulated under growing conditions where an essential nutrient is found in limiting concentrations. Polyhydroxyalkanoates (PHAs) are formed as intracellular granules and can be accumulated up to 80% by weight of the cell mass. The most common form of polyhydroxyalkanoate (PHA) produced by said microorganisms is poly-hydroxy-3-butyrate (P3HB). Other forms of polyhydroxyalkanoates (PHAs), which may include co-polymers, are formed in the presence of specific structurally related precursors or substrates.

In accordance with a preferred embodiment of the present disclosure, said lignocellulosic hydrolyzate is the aqueous phase that derives from the hydrolysis of a lignocellulosic biomass which can be selected, for example, from:
scraps, residues and waste of products deriving from crops specifically cultivated for energy purpose such as, for example, miscanthus, panicum (Panicum virgatum), common reed (Arundo donax);
scraps, residues and waste from products deriving from agriculture such as, for example, guayule, corn, soy, cotton, linseed, rapeseed, sugar cane, palm oil, poplar, alder, birch, residues deriving from the oil palm tree [palm leaf, trunks, leaf midribs, empty fruits of palm oil (EFB—“Empty Fruit Bunches”)], wheat straw, rice straw, corn stalks, cotton stems, sorghum, bagasse (for example, sugar cane bagasse);
scraps, residues and waste from products deriving from forestation or forestry including scraps, residues and waste deriving from such products or their processing;
scraps from agri-food products intended for human nutrition or animal husbandry;
residues, not chemically treated, from the paper industry;
waste from the separate collection of municipal solid waste (for example, urban waste of vegetable origin, paper);
algae such as, for example, microalgae or macroalgae, in particular macroalgae.

In accordance with a particularly preferred embodiment of the present disclosure, said lignocellulosic biomass can be selected, for example, from scraps, residues and waste deriving from miscanthus, panic (Panicum virgatum), common reed (Arundo donax), guayule, poplar, alder, birch, sorghum, corn stalks, cotton stems, sugar cane bagasse, leaf mibrids, empty palm oil fruits (EFB—“Empty Fruit Bunches”), wheat straw, rice straw, cotton stems.

Preferably, said lignocellulosic biomass can be subjected to a preliminary grinding process before being subjected to hydrolysis. Preferably, said lignocellulosic biomass can be ground to obtain particles having a diameter between 0.1 mm and 10 mm, more preferably between 0.5 mm and 4 mm. Particles having a diameter of less than 1 mm are particularly preferred.

For the purpose of the present disclosure, the hydrolysis of the lignocellulosic biomass can be carried out according to anyone of the methods known in the art. Non-limiting examples of these methods are:
heat treatment known as “steam explosion”, followed by enzymatic hydrolysis, as described, for example, in the international patent application WO 2012/042544;
treatment in the presence of diluted acids, for example diluted sulphuric acid, followed by enzymatic hydrolysis, as described, for example, by Humbrid D. et al., in “Technical Report Nrel/Tp-5100-47764 (May 2011);
treatment in the presence of organic acids, for example 2-naphthalene-sulfonic acid, followed by enzymatic hydrolysis, as described, for example, in the international patent application WO 2010/046051; or methanesulfonic acid, followed by enzymatic hydrolysis, as described, for example, in the international patent application WO 2016/062753 in the name of the Applicant;
treatment in the presence of bases, for example, sodium hydroxide, followed by enzymatic hydrolysis, as described, for example, in the international patent application WO 2014/144588.

Said enzymatic hydrolysis can be carried out according to methods known in the art as described, for example, in the American patents U.S. Pat. Nos. 5,628,830, 5,916,780, 6,090,595, using commercial enzymes such as, for example, Celluclast 1.5 L (Novozymes), Econase CE (Rohm Enzymes), Spezyme (Genecor), Novozym 188 (Novozymes), used singly or mixed together.

From said hydrolysis a mixture is obtained comprising a solid residue (i.e. solid phase) and a lignocellulosic hydrolyzate (i.e. aqueous phase). Said mixture is subjected to filtration or centrifugation in order to obtain a solid residue (i.e. solid phase) and a lignocellulosic hydrolyzate (i.e. aqueous phase).

Said solid residue (i.e. solid phase) comprises lignin and said lignocellulosic hydrolyzate (i.e. aqueous phase) comprises at least one sugar having from 5 carbon atoms (C5) to 6 carbon atoms (C6), more preferably xylose and glucose.

In accordance with a preferred embodiment of the present disclosure, said lignocellulosic hydrolyzate, before being used, can be subjected to pasteurisation at a temperature between 60° C. and 90° C., preferably between 70° C. and 85° C., for a time between 10 minutes and 1 hour, preferably between 15 minutes and 50 minutes.

In accordance with a preferred embodiment of the present disclosure, in said step (a), said microorganism capable of using sugars with six carbon atoms (C6) and organic acids, can be selected, for example, from the microorganisms belonging to following genera: Cupriavidus, Pseudomonas, Bacillus, Ralstonia, Halomonas, Alcaligenes, Escherichia, preferably Cupriavidus, Pseudomonas, Bacillus.

For the purpose of the present disclosure, said microorganism capable of using sugars with six carbon atoms (C6) or organic acids, can be either wild-type or genetically modified.

Organic acids which can be used by said microorganism are, for example, acetic acid, formic acid, butyric acid, propionic acid, valeric acid, lactic acid, especially acetic acid.

In accordance with a preferred embodiment of the present disclosure, said process can comprise, before said step (a), a propagation step of said microorganism capable of using sugars with six carbon atoms (C6) or organic acids, obtaining an inoculum.

In order to obtain said inoculum, said microorganism capable of using sugars with six carbon atoms (C6) or organic acids, is fed to a fermentation device in the presence of a culture medium usually used for the purpose which may include, in addition to sugars, various nutrients such as, for example, nitrogen, potassium phosphate, sodium phosphate, potassium sulphate, magnesium sulphate, citric acid, other salts, vitamins, microelements and, when the microorganism reaches a cell concentration (dry weight) greater than or equal to 3 g/L, preferably between 5 g/L and 8 g/L, the inoculum can be fed to said first fermentation device. In order to obtain said inoculum, said propagation step can be carried out:
at a temperature between 20° C. and 45° C., preferably between 25° C. and 40° C.; and/or
for a time between 30 minutes and 30 hours, preferably between 4 hours and 24 hours; and/or
at an air flow rate, automatically fed, between 30 L/Lh and 300 L/Lh, preferably between 60 L/Lh and 180 L/Lh; and/or
at a pH between 6 and 8, preferably between 6.5 and 7.5 (in order to maintain the pH in the desired ranges, an aqueous solution of at least one inorganic base such as, for example, sodium hydroxide, potassium hydroxide, calcium hydroxide, magnesium hydroxide, ammonium hydroxide, or mixtures thereof, preferably potassium hydroxide, ammonium hydroxide, or at least one inorganic acid such as, for example, phosphoric acid, sulphuric acid, hydrochloric acid, or mixtures thereof, preferably sulfuric acid, in a quantity such as to obtain the desired pH, can be added).

In accordance with a preferred embodiment of the present disclosure, in said step (a), in addition to the lignocellulosic hydrolyzate, to said first fermentation device a culture medium usually used for the purpose which can comprise, in addition to sugars, various nutrients such as, for example, nitrogen, potassium phosphate, sodium phosphate, potassium sulphate, magnesium sulphate, citric acid, other salts, vitamins, microelements, can be fed.

In order to avoid substrate inhibition, before said step (a), the lignocellulosic hydrolyzate can be diluted with water so as to have a final glucose concentration in said first fermentation device between 5 g/l and 50 g/l, preferably between 10 g/l and 30 g/l.

Subsequently, in order to keep the quantity of glucose constant in said first fermentation device during the fermentation itself, the hydrolyzate, as it is or concentrated, is fed to said first fermentation device, in order to grow said microorganism capable of use sugars with six carbon atoms (C6) and organic acids, according to a well-defined feeding strategy, which is important for controlling cell growth, biosynthesis and the composition of the polyhydroxyalkanoates (PHAs) produced. Said feeding strategy is known in the art and is described, for example, by Yamané T. et al., in “Fed-batch Techniques in Microbial Processes”, “Bioprocess Parameter Control. Advances in Biochemical Engineering Biotechnology” (1984), Vol 30, pg. 147-194, Springer, Berlin, Heidelberg.

In accordance with a preferred embodiment of the present disclosure, in said step (a), said microorganism capable of using sugars with six carbon atoms (C6) and organic acids, can be used at an initial cell concentration (dry weight) between 0.1 g/L and 2 g/L, preferably between 0.2 g/L and 1 g/L.

In accordance with a preferred embodiment of the present disclosure, in said step (a), the fermentation in said first fermentation device can be carried out at a temperature between 20° C. and 45° C., preferably between 25° C. and 40° C.

In accordance with a preferred embodiment of the present disclosure, in said step (a), the fermentation in said first fermentation device can be carried out for a time between 1 day and 6 days, preferably between 1.5 days and 5 days.

In accordance with a preferred embodiment of the present disclosure, in said step (a), the fermentation in said first fermentation device can be carried out at a pH between 6 and 8, preferably between 6.5 and 7.5. In order to maintain the pH in the desired ranges, an aqueous solution of at least one inorganic base such as, for example, sodium hydroxide, potassium hydroxide, calcium hydroxide, magnesium hydroxide, ammonium hydroxide, or mixtures thereof, preferably potassium hydroxide, ammonium hydroxide, or at least one inorganic acid such as, for example, phosphoric acid, sulphuric acid, hydrochloric acid, or mixtures thereof, preferably sulphuric acid, in a quantity such as to obtain the desired pH, can be added.

In accordance with a preferred embodiment of the present disclosure, in said step (a), the fermentation in said first fermentation device can be carried out at an air flow rate between 30 L/Lh and 300 L/Lh, preferably between 50 L/Lh and 180 L/Lh. For this purpose, the air inside said first fermentation device can be automatically fed so as to maintain, together with the increase in agitation, the oxygen saturation levels in the culture medium at values between 10% and 55%, preferably between 18% and 30%.

It should be noted that when the cell concentration increases, the oxygen concentration inside the fermentation device begins to decrease and a cascade control is then activated, which increases the agitation or modulates the air flow.

In accordance with a preferred embodiment of the present disclosure, in said step (a), the fermentation in said first fermentation device can be carried out in one or more steps, in a batch mode, in a fed-batch mode, in continuous mode, preferably in a fed-batch mode.

At the end of fermentation, in said step (b), the separation to which said first fermentation broth is subjected in order to recover said aqueous suspension of cellular biomass comprising said at least one polyhydroxyalkanoate (PHA) and said aqueous phase comprising sugars with five carbon atoms (C5) (said aqueous phase possibly containing suspended solids, for example, cells of the microorganism used in the fermentation, or particulate resulting from the deterioration of the equipment used in the process, or from the precipitation of salts), can be carried out by methods known in the art such as, for example, filtration, filter pressing, microfiltration or ultrafiltration, centrifugation.

The extraction of said at least one polyhydroxyalkanoate (PHA) from cellular biomass can be carried out according to methods known in the art as described, for example, in U.S. Pat. Nos. 4,324,907 and 5,110,980.

In accordance with a preferred embodiment of the present disclosure, the aqueous phase obtained in said step (b), before being fed to the second fermentation device [step (c)] or to the third fermentation device [step (d)], can be subjected to pasteurization at a temperature between 60° C. and 90° C., preferably between 70° C. and 85° C., for a time between 30 minutes and 1 hour, preferably between 40 minutes and 50 minutes.

In accordance with a preferred embodiment of the present disclosure, in said step (c), in addition to the aqueous phase obtained in said step (b), to said second fermentation device a culture medium comprising sugars and urea as a source of nitrogen can be fed and, when the microorganism reaches a cellular concentration (dry weight) greater than or equal to 1 g/L, preferably between 5 g/L and 8 g/L, the second fermentation broth (inoculum) can be fed to said third fermentation device [step (d)]. The propagation in order to obtain said inoculum, in said second fermentation device, can be carried out:
at a temperature between 20° C. and 40° C., preferably between 25° C. and 35° C.; and/or
for a time between 1 hour and 30 hours, preferably between 4 hours and 24 hours; and/or
at an air flow rate, automatically fed, between 1 L/Lh and 60 L/Lh, preferably between 10 L/Lh and 30 L/Lh; and/or
at a pH of between 4 and 7, preferably between 4.5 and 6.5 (in order to maintain the pH in the desired ranges, an aqueous solution of at least one inorganic base such as, for example, sodium hydroxide, potassium hydroxide, calcium hydroxide, magnesium hydroxide, or mixtures thereof, preferably potassium hydroxide, or at least one inorganic acid such as, for example, phosphoric acid, sulphuric acid, hydrochloric acid, or mixtures thereof, preferably sulphuric acid, in a quantity such as to obtain the desired pH, can be added).

In accordance with a preferred embodiment of the present disclosure, in said step (c) and in said step (d), said microorganism capable of using both sugars with five carbon atoms (C5) and sugars with 6 carbon atoms (C6) can be selected, for example, from the microorganisms belonging to the following genera: Saccharomyces, Zygosaccharomyces, Candida, Hansenula, Kluyveromyces, Debaromyces, Nadsonias, Lipomyces, Torulopsis, Kloeckera, Pichia, Schizosaccharomyces, Trigonopsis, Brettanomyces, Cryptococcus, Trichosporon, Aureobasidium, Lipomyces, Phaffia, Rhodotorula, Yarrowia, Schwanniomyces, preferbly Saccharomyces.

For the purpose of the present disclosure, said microorganism capable of using both sugars with five carbon atoms (C5) and sugars with 6 carbon atoms (C6), can be either wild-type or genetically modified and capable of metabolize glucose and/or xylose simultaneously.

In accordance with a preferred embodiment of the present disclosure, in said step (d), said microorganism capable of using both sugars with five carbon atoms (C5) and sugars with 6 carbon atoms (C6), can be directly fed to said third fermentation device (“direct pitching”).

In accordance with a preferred embodiment of the present disclosure, said process can comprise, prior to said step (d), a propagation step of said microorganism capable of using both sugars with five carbon atoms (C5) and sugars with six carbon atoms (C6) obtaining an inoculum.

In order to obtain said inoculum, said microorganism capable of using both sugars with five carbon atoms (C5) and sugars with six carbon atoms (C6), is fed to a fermentation device in the presence of a culture medium comprising sugars and urea as a source of nitrogen and, when the microorganism reaches a cell concentration (dry weight) greater than or equal to 1 g/L, preferably between 5 g/L and 8 g/L, the inoculum can be fed to said third fermentation device. In order to obtain said inoculum, the propagation can be carried out:
at a temperature between 20° C. and 40° C., preferably between 25° C. and 35° C.; and/or
for a time between 1 hour and 30 hours, preferably between 4 hours and 24 hours; and/or
at an air flow rate, automatically fed, between 1 L/Lh and 60 L/Lh, preferably between 10 L/Lh and 30 L/Lh; and/or
at a pH between 4 and 7, preferably between 4.5 and 6.5 (in order to maintain the pH in the desired ranges, an aqueous solution of at least one inorganic base such as, for example, sodium hydroxide, potassium hydroxide, calcium hydroxide, magnesium hydroxide, or mixtures thereof, preferably potassium hydroxide, or at least one inorganic acid such as, for example, phosphoric acid, sulphuric acid, hydrochloric acid, or mixtures thereof, preferably sulphuric acid, in a quantity such as to obtain the desired pH, can be added).

In accordance with a preferred embodiment of the present disclosure, in said step (d), in addition to the aqueous phase obtained in said step (b) and, optionally, to the lignocellulosic hydrolyzate, a medium can be fed to said third fermentation device culture including sugars and urea as a source of nitrogen.

In accordance with a further embodiment of the present disclosure, the aqueous phase obtained in said step (b), can be joined to at least a part of said lignocellulosic hydrolyzate before being fed to said third fermentation device [step (d)].

In accordance with a further embodiment of the present disclosure, the aqueous phase obtained in said step (b) can be joined to at least a part of said solid residue (i.e. solid phase) obtained after hydrolysis of the lignocellulosic biomass, before being fed to said third fermentation device [step (d)].

In accordance with a further embodiment of the present disclosure, the aqueous phase obtained in said step (b) can be joined to at least a part of said lignocellulosic hydrolyzate and to at least a part of said solid residue (i.e. solid phase) obtained after hydrolysis of the lignocellulosic biomass, before being fed to said third fermentation device.

In accordance with a further embodiment of the present disclosure, at least a part of the aqueous phase obtained in said step (b), can be fed to the hydrolysis of the lignocellulosic biomass.

In accordance with a preferred embodiment of the present disclosure, in said step (d), said microorganism capable of using both sugars with 5 carbon atoms (C5) and sugars with 6 carbon atoms (C6), can be used at an initial cell concentration (dry weight) between 0.1 g/L and 2 g/L, preferably between 0.2 g/L and 1 g/L.

In accordance with a preferred embodiment of the present disclosure, in said step (d), the fermentation in said third fermentation device can be carried out at a temperature between 20° C. and 40° C., preferably between 25° C. and 35° C.

In accordance with a preferred embodiment of the present disclosure, in said step (d), the fermentation in said third fermentation device can be carried out for a time between 1 day and 6 days, preferably between 1.5 days and 4 days.

In accordance with a preferred embodiment of the present disclosure, in said step (d), the fermentation in said third fermentation device can be carried out at a pH between 4 and 7, preferably between 4.5 and 6.5. In order to maintain the pH in the desired ranges, an aqueous solution of at least one inorganic base such as, for example, sodium hydroxide, potassium hydroxide, calcium hydroxide, magnesium hydroxide, or mixtures thereof, preferably potassium hydroxide, or at least one inorganic acid such as, for example, phosphoric acid, sulphuric acid, hydrochloric acid, or mixtures thereof, preferably sulphuric acid, in a quantity such as to obtain the desired pH, can be added.

In accordance with a preferred embodiment of the present disclosure, the fermentation in said third fermentation device can be carried out in one or more steps, in a batch mode, in a fed-batch mode, in continuous mode, preferably in a batch mode.

At the end of fermentation [step (e)], said third fermentation broth is subjected to separation by operating according to techniques known in the art such as, for example, distillation, centrifugation, extraction, preferably distillation, obtaining bioethanol.

Distillation can be carried out according to methods known in the art as described, for example, in “Ethanol distillation: the fundamentals” (1999), Chapter 18, pg. 269-288, Katzen R., Madson P. W. and Moon G. D., Jr KATZEN International, Inc., Cincinnati, Ohio, USA.

### BRIEF DESCRIPTION OF THE DRAWING

The present disclosure will now be illustrated in greater detail through an embodiment with reference to FIG. 1 below.

FIG. 1 schematizes an embodiment of the present disclosure. For this purpose, the lignocellulosic biomass (e.g., previously ground lignocellulosic biomass) is subjected to hydrolysis (operating according to one of the methods known in the art reported above) obtaining a mixture comprising a solid residue (i.e. solid phase) and a lignocellulosic hydrolyzate (i.e. aqueous phase). Said mixture is subjected to filtration or centrifugation (not represented in FIG. 1) obtaining a solid residue (i.e. solid phase) and a lignocellulosic hydrolyzate (i.e. aqueous phase). At least a part of said lignocellulosic hydrolyzate is fed to a first fermentation device obtaining a first fermentation broth. Said first fermentation broth is subjected to separation (e.g., by centrifugation) obtaining an aqueous suspension of cellular biomass from which at least one polyhydroxyalkanoate (PHA) and an aqueous phase are extracted. A part of said aqueous phase and, optionally, a part of said lignocellulosic hydrolyzate (indicated with dashed line in FIG. 1) is/are fed to a third fermentation device obtaining a third fermentation broth which is subjected to distillation obtaining bioethanol.

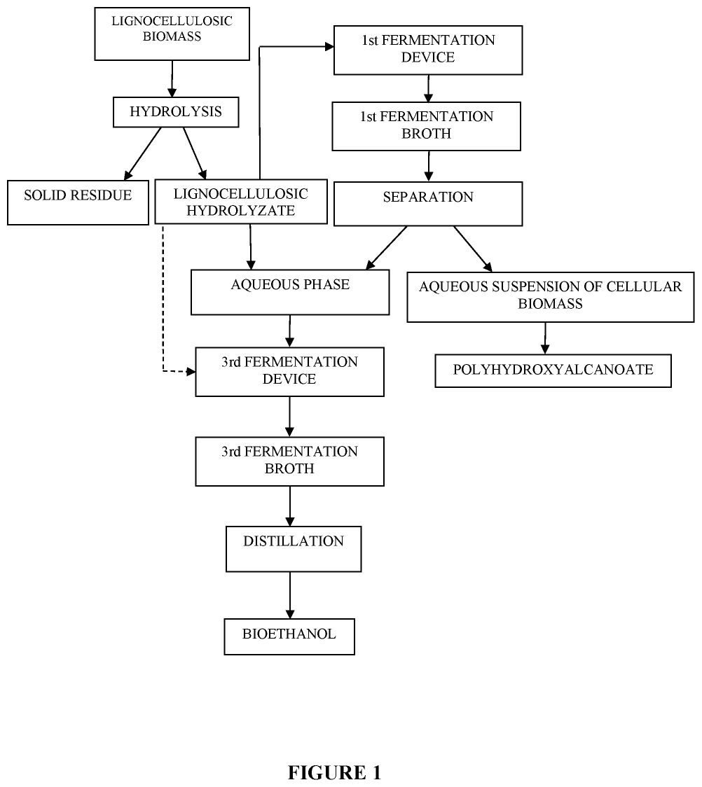

### DETAILED DESCRIPTION OF THE DISCLOSURE

In order to better understand the present disclosure and to put it into practice, some illustrative and non-limiting examples thereof are shown below.

### Example 1 (Disclosure)

Production of poly-hydroxy-3-butyrate (P3HB)

The lignocellulosic hydrolyzate obtained from poplar used in the examples was pasteurised, at 80° C., for 45 minutes. Subsequently, the sugar and organic acid content of said lignocellulosic hydrolyzate was determined by high performance liquid chromatography (HPLC) using an end-capped Metacarb 67H column (300 mm×6.5 mm; 1/pk) by Agilent, equipped with a photodiode UV detector and refractive index (RI) detector and with 5 mM phosphoric acid mobile phase in water, operating under the following conditions:
pump flow: 0.8 mL/min (5 mM sulphuric acid);
injection volume: 20 μL;
column oven temperature: 45° C.;
RI temperature detector: 35° C.;
UV detector wavelengths: 210 nm and 280 nm;
analysis time: 35 minutes.

The lignocellulosic hydrolyzate was found to comprise 44.45 g/L of glucose, 20.51 g/L of xylose and 4.46 g/L of acetic acid.

0.6 g/L di Na₂HPO₄·7 H₂O, 2.0 g/L KH₂PO₄, 2.0 g/L (NH₄)₂SO₄, 0.2 g/L MgSO₄·7 H₂O, 20 mg/L CaCl₂, 10 g/L of glucose and 1 g/L of yeast extract, were placed in a 500 ml flask, equipped with a magnetic stirrer, obtaining a mixture with was sterilized in autoclave at 120° C., for 20 minutes. At the end of the sterilisation, 1 ml/L of a trace metal solution having the following composition was added to said mixture: 0.2 mg/l FeSO₄·7 H₂O, 0.6 mg/L H₃BO₃, 1.3 mg/L ZnSO₄, 0.6 mg/l (NH₄)₆Mo₇O₂₄·6 H₂O, previously sterilised by filtration with filters of 0.2 microns. Subsequently, the mixture obtained was brought to room temperature (25° C.) and inoculated with Cupriavidus necator cells which were left to grow, for 24 hours, at 30° C., under stirring (200 rpm) until a concentration of cellular biomass having an optical density (OD₆₀₀) equal to 15 [3 g/L (dry weight)] was obtained.

Fermentation in the first fermentation device with Cupriavidus necator was carried out in a 2 L bioreactor, operating under the following conditions:
0.2 L of the aforementioned lignocellulosic hydrolyzate suitably diluted in water so as to have an initial glucose concentration equal to 10 g/L;
0.6 g/L of Na₂HPO₄·7 H₂O, 2.0 g/L KH₂PO₄, 2.0 g/L (NH₄)₂SO₄, 0.2 g/L MgSO₄·7 H₂O, 20 mg/L CaCl₂), 10 g/L of glucose, 1 g/L of yeast extract and 1 ml/L of a trace metal solution having the following composition: 0.2 mg/l FeSO₄·7 H₂O, 0.6 mg/L H₃BO₃, 1.3 mg/L ZnSO₄, 0.6 mg/l (NH₄)₆Mo₇O₂₄·6 H₂O (all previously sterilised operating as described above);
supplied air: flow rate equal to 60 L/Lh;
temperature: 30° C.;
operating pH equal to 7, maintained by adding, when necessary, a few drops of a solution of potassium hydroxide (KOH) 5 M and sulphuric acid (H₂SO₄) 10% (v/v);
agitation equal to 600 rpm-900 rpm, modulated with the air flow rate in order to maintain the concentration of dissolved oxygen (DO₂) above 20% with respect to the saturation value;
initial volume: 0.7 litres;
inoculation of Cupriavidus necator obtained as described above diluted to 10% (v/v) with the culture medium used for fermentation, in order to start fermentation with a concentration of cellular biomass equal to 0.3 g/L (weight dry).

The fermentation was carried out in fed-batch mode for 3 days by feeding, on the second and third day, a total quantity equal to 0.33 L of concentrated 2× lignocellulosic hydrolyzate, in order to restore the concentration of glucose. Cell growth was monitored by sampling the culture medium every 3 hours. The sample taken (5 ml) was centrifuged at 4000 rpm, for 10 minutes, at room temperature (25° C.), in calibrated test tubes. The pellet obtained was washed with demineralised water, centrifuged again and dried at 65° C., up to constant weight. Cell concentration was calculated as the weight difference between the sample tube and the empty tube. The discarded supernatant was used to monitor the concentration of sugars and organic acids by chromatographic analysis as described above.

At the end of the fermentation, the first fermentation broth was subjected to separation by centrifugation at 6000 rpm, for 10 minutes, obtaining 18 g/L of cellular biomass and an aqueous phase. The cellular biomass obtained was washed with water, frozen at −20° C., lyophilised and subjected to extraction. For this purpose, the lyophilised cellular biomass was washed with ethanol (0.5 L), at 50° C., for two hours in a 1 L flask, rotating in a rotavapor, at 100 rpm. The suspension was filtered with a cellulose filter and placed in a 1 L reactor, equipped with a mechanical stirrer, in the presence of chloroform (0.4 L) at a temperature of 60° C., for 4 hours, at 100 rpm. At the end of the extraction, the solution obtained was centrifuged in order to remove the suspended solids: the liquid obtained was concentrated and precipitated with cold ethanol at −20° C., obtaining 12 g/L of poly-hydroxy-3-butyrate (P3HB), equal to 66% of the dry cell weight, with a yield equal to 0.32 g P3HB/g substrate consumed, with the complete consumption of the acetic acid contained in the starting lignocellulosic hydrolyzate.

The aqueous phase containing glucose and xylose and detoxified by acetic acid, was used in the fermentation with bioethanol.

The poly-hydroxy-3-butyrate (P3HB) obtained was subjected to characterisation by operating as follows.

The ¹H-HMR spectrum was recorded by means of a nuclear magnetic resonance spectrometer mod. Bruker Avance 400, using deuterated chloroform (CDCl₃), at 25° C. and tetramethylsilane (TMS) as an internal standard. For this purpose, a poly-hydroxy-3-butyrate (P3HB) solution was used having a concentration equal to 10% by weight with respect to the total weight of the solution.

¹H-NMR (CDCl₃, δ ppm): 5.3 (s, 1H—O—CH), 2.7-2.4 (m, 2H—CH₂—CO), 1.3 (d, 3H—CH₃).

The determination of the molecular weight (MW) of the obtained poly-hydroxy-3-butyrate (P3HB) was carried out by GPC (“Gel Permeation Chromatography”), using the Waters® Alliance® GPC/V 2000 System of Waters Corporation which uses two detection lines: refractive index (RI) and viscometer, operating under the following conditions:
two PL gel Mixed-B columns;
solvent/eluent: chloroform;
flow: 1 ml/min;
temperature: 35° C.;
calculation of the molecular mass: Universal Calibration method.

The weight average molecular weight (M_{w}) and the polydispersion index (PDI) corresponding to the M_{w}/Mₙ ratio (Mₙ=number average molecular weight) are shown:
M_{w}: 204000 Dalton;
polydispersion index (PDI): 4.7.

The DSC (“Differential Scanning Calorimetry”) thermal analysis, in order to determine the melting temperature (Tₘ) and the melting enthalpy (ΔHₘ) of the poly-hydroxy-3-butyrate (P3HB) obtained, was carried out by a Perkin Elmer Pyris differential scanning calorimeter. For this purpose, 10 mg of pulverised poly-hydroxy-3-butyrate (P3HB) were hermetically sealed inside a perforated aluminium crucible: the sample thus prepared was subjected to DSC (Differential Scanning Calorimetry) thermal analysis and to a first heating and cooling cycle which is essential to cancel the thermal history. Subsequently, the sample was subjected to a heating cycle through which the melting temperature (Tₘ) e and the melting enthalpy (ΔHₘ) was measured.

The heating and cooling cycle and the subsequent heating cycle were conducted as follows:
heating: from 0° C. to 190° C. at a speed of 10° C./min;
cooling: from 190° C. to 0° C. at a speed of 20° C./min;
isotherm: at 0° C. for 1/min;
heating: from 0° C. to 190° C. at a speed of 10° C./min.

The poly-hydroxy-3-butyrate (P3HB) was found to have a melting temperature (Tₘ) equal to 177.3° C. and a melting enthalpy (ΔH) equal to 87.2 J/g (corresponding to a crystallinity equal to approximately 60%).

The thermogravimetric analysis (TGA) was carried out using the Q500 Thermal Analysis tool (TA Instruments, New Castle, DE, USA). For this purpose, 5 mg of poly-hydroxy-3-butyrate (P3HB) was placed in an aluminium crucible, pre-heated to 30° C. and subsequently heated, at a rate of 20° C./min, up to 600° C. The results of thermogravimetric analysis (TGA) showed a degradation temperature of poly-hydroxy-3-butyrate (P3HB) at 302.3° C. and a residue at 600° C. equal to 0%. The onset of degradation took place at approximately 253° C., temperature at which the residual weight of the sample was equal to 99.6% of the initial weight, whilst at approximately 316° C. the residual weight of the initial weight was equal to 0.75%.

### Example 2 (Comparative)

### Production of Bioethanol

Fermentation in the third fermentation device with Saccharomyces cerevisiae cells was carried out in a 2 L bioreactor, operating under the following conditions:
1.1 L of lignocellulosic hydrolyzate as described above;
1.5 g of urea;
temperature: 32° C.;
operating pH equal to 5.5, maintained by adding, when necessary, a few drops of a solution of sodium hydroxide (NaOH) 5 M and sulphuric acid (H₂SO₄) 10% (v/v);
agitation equal to 80 rpm;
initial volume: 1.1 litres;
0.5 g/L of Saccharomyces cerevisiae (“direct pitching”).

Fermentation was carried out in batch mode for 2 days and cell growth was monitored by cell count under an optical microscope.

At the end of the fermentation, the third culture broth was subjected to distillation obtaining 27 g/L of bioethanol.

### Example 3 (Disclosure)

### Production of Bioethanol

Fermentation in the third fermentation device with Saccharomyces cerevisiae cells was carried out in a 2 L bioreactor, operating under the following conditions:
0.77 L of aqueous phase obtained from the first fermentation broth comprising 20 g/L of xylose and 10 g/L of glucose mixed with 0.33 L of lignocellulosic hydrolyzate so as to restore the glucose concentration to 44.45 g/L; 1.5 g/L of urea;
temperature: 32° C.;
operating pH equal to 5.5, maintained by adding, when necessary, a few drops of a solution of sodium hydroxide (NaOH) 5 M and sulphuric acid (H₂SO₄) 10% (v/v);
agitation equal to 80 rpm;
initial volume: 1.1 litres;
0.5 g/L of Saccharomyces cerevisiae (“direct pitching”).

Fermentation was carried out in batch mode for 2 days and cell growth was monitored by cell count under an optical microscope.

At the end of the fermentation, the third culture broth was subjected to distillation obtaining 30 g/L of bioethanol.

1. An integrated process for the production of polyhydroxyalkanoates (PHAs) and bioethanol from lignocellulosic hydrolyzate, the process including the following steps:
(a) feeding at least a part of said lignocellulosic hydrolyzate to a first fermentation device in the presence of at least one microorganism capable of using sugars with six carbon atoms (C6) and organic acids, obtaining a first fermentation broth;
(b) subjecting the first fermentation broth obtained in said step (a) to separation obtaining an aqueous suspension of cellular biomass comprising at least one polyhydroxyalkanoate (PHA) and an aqueous phase comprising sugars with five carbon atoms (C5) in a quantity greater than or equal to 10 g/L;
(c) optionally, feeding at least a part of the aqueous phase obtained in said step (b) to a second fermentation device in the presence of at least one microorganism capable of using both sugars with five carbon atoms (C5) and sugars with six carbon atoms (C6), obtaining a second fermentation broth (inoculum);
(d) feeding at least a part of the aqueous phase obtained in said step (b) and, optionally, the second fermentation broth (inoculum) obtained in said step (c) and/or at least a part of said lignocellulosic hydrolyzate, to a third fermentation device in the presence of at least one microorganism capable of using both sugars with five carbon atoms (C5) and sugars with six carbon atoms (C6), obtaining a third fermentation broth;
(e) subjecting said third fermentation broth to separation obtaining bioethanol.

2. The integrated process for the production of polyhydroxyalkanoates (PHAs) and bioethanol in accordance with claim 1, wherein said lignocellulosic hydrolyzate is the aqueous phase that derives from the hydrolysis of a lignocellulosic biomass selected from:
scraps, residues and waste of products deriving from crops specifically cultivated for energy purpose such as miscanthus, panicum (Panicum virgatum), common reed (Arundo donax);
scraps, residues and waste from products deriving from agriculture such as guayule, corn, soy, cotton, linseed, rapeseed, sugar cane, palm oil, poplar, alder, birch, residues deriving from the oil palm tree [palm leaf, trunks, leaf midribs, empty fruits of palm oil (EFB—“Empty Fruit Bunches”)], wheat straw, rice straw, corn stalks, cotton stems, sorghum, bagasse (for example, sugar cane bagasse);
scraps, residues and waste from products deriving from forestation or forestry including scraps, residues and waste deriving from such products or their processing;
scraps from agri-food products intended for human nutrition or animal husbandry;
residues, not chemically treated, from the paper industry;
waste from the separate collection of municipal solid waste (such as urban waste of vegetable origin, paper);
algae such as microalgae or macroalgae;
said lignocellulosic biomass is selected from the group consisting of scraps, residues and waste deriving from miscanthus, panic (Panicum virgatum), common cane (Arundo donax), guayule, poplar, alder, birch, sorghum, corn stalks, sugar cane bagasse, leaf mibrids, empty palm oil fruits (EFB—“Empty Fruit Bunches”), wheat straw, rice straw, and cotton stems.

3. The integrated process for the production of polyhydroxyalkanoates (PHAs) and bioethanol according to claim 1, wherein said lignocellulosic hydrolyzate, before being used, is subjected to pasteurisation at a temperature between 60° C. and 90° C., for a time between 10 minutes and 1 hour.

4. The integrated process for the production of polyhydroxyalkanoates (PHAs) and bioethanol in accordance with claim 1, wherein in said step (a), said microorganism capable of using sugars with six carbon atoms (C6) and organic acids, is selected from the microorganisms belonging to the following genera: Cupriavidus, Pseudomonas, Bacillus, Ralstonia, Halomonas, Alcaligens, Escherichia.

5. The integrated process for the production of polyhydroxyalkanoates (PHAs) and bioethanol according to claim 1, wherein said process comprises, before said step (a), a propagation step of said microorganism capable of using sugars with six atoms of carbon (C6) and organic acids, obtaining an inoculum.

6. The integrated process for the production of polyhydroxyalkanoates (PHAs) and bioethanol according to claim 1, wherein in said step (a), in addition to the lignocellulosic hydrolyzate, to said first fermentation device a culture medium usually used for the purpose which comprise, in addition to sugars, various nutrients such as nitrogen, potassium phosphate, sodium phosphate, potassium sulphate, magnesium sulphate, citric acid, other salts, vitamins, microelements, is fed.

7. The integrated process for the production of polyhydroxyalkanoates (PHAs) and bioethanol according to claim 1, wherein in said step (a) said microorganism capable of using sugars with 6 carbon atoms (C6) and organic acids, is used at an initial cell concentration (dry weight) between 0.1 g/L and 2 g/L.

8. The integrated process for the production of polyhydroxyalkanoates (PHAs) and bioethanol according to claim 1, wherein in said step (a), the fermentation in said first fermentation device is carried out:
at a temperature of between 20° C. and 45° C.; and/or
for a time between 1 day and 6 days; and/or
at a pH between 6 and 8; and/or
at an air flow rate between 30 L/Lh and 300 L/Lh; and/or
in one or more steps, in a batch mode, in a fed-batch mode, in continuous mode.

9. The integrated process for the production of polyhydroxyalkanoates (PHAs) and bioethanol according to claim 1, wherein the aqueous phase obtained in said step (b), before being fed to the second fermentation device [step (c)] or to the third fermentation device [step (d)], is subjected to pasteurisation at a temperature between 60° C. and 90° C., for a time between 30 minutes and 1 hour.

10. The integrated process for the production of polyhydroxyalkanoates (PHAs) and bioethanol according to claim 1, wherein in said step (c), in addition to the aqueous phase obtained in said step (b), to said second fermentation device a culture medium comprising sugars and urea as a source of nitrogen is fed and, when the microorganism reaches a cell concentration (dry weight) greater than or equal to 3 g/L, the second fermentation broth (inoculum) is fed to said third fermentation device [step (d)].

11. The integrated process for the production of polyhydroxyalkanoates (PHAs) and bioethanol according to claim 1, wherein in said step (c) and in said step (d), said microorganism capable to use both sugars with five carbon atoms (C5), and sugars with 6 carbon atoms (C6) is selected from the microorganisms belonging to the following genera: Saccharomyces, Zygosaccharomyces, Candida, Hansenula, Kluyveromyces, Debaromyces, Nadsonias, Lipomyces, Torulopsis, Kloeckera, Pichia, Schizosaccharomyces, Trigonopsis, Brettanomyces, Cryptococcus, Trichosporon, Aureobasidium, Lipomyces, Phaffia, Rhodotorula, Yarrowia, Schwanniomyces.

12. The integrated process for the production of polyhydroxyalkanoates (PHAs) and bioethanol according to claim 1, wherein in said step (d), said microorganism capable to use both sugars with five carbon atoms (C5) and sugars with 6 carbon atoms (C6), is directly fed to said third fermentation device (“direct pitching”).

13. The integrated process for the production of polyhydroxyalkanoates (PHAs) and bioethanol according to claim 1, wherein said process comprises, before said step (d), a propagation step of said microorganism capable of using both five-step sugars carbon atoms (C5), and sugars with six carbon atoms (C6) obtaining an inoculum.

14. The integrated process for the production of polyhydroxyalkanoates (PHAs) and bioethanol according to claim 1, wherein in said step (d), in addition to the aqueous phase obtained in said step (b) and, optionally, to the lignocellulosic hydrolyzate, a culture medium comprising sugars and urea as a nitrogen source is fed to said third fermentation device.

15. The integrated process for the production of polyhydroxyalkanoates (PHAs) and bioethanol according to claim 1, wherein the aqueous phase obtained in said step (b), is joined to at least a part of said lignocellulosic hydrolyzate before being fed to said third fermentation device [step (d)].

16. The integrated process for the production of polyhydroxyalkanoates (PHAs) and bioethanol according to claim 1, wherein the aqueous phase obtained in said step (b) is joined to at least a part of said solid residue (i.e. solid phase) obtained after hydrolysis of the lignocellulosic biomass, before being fed to said third fermentation device [step (d)].

17. The integrated process for the production of polyhydroxyalkanoates (PHAs) and bioethanol according to claim 1, wherein the aqueous phase obtained in said step (b) is joined to at least a part of said lignocellulosic hydrolyzate and to at least a part of said solid residue (i.e., solid phase) obtained after hydrolysis of the lignocellulosic biomass, before being fed to said third fermentation device.

18. The integrated process for the production of polyhydroxyalkanoates (PHAs) and bioethanol in accordance with claim 1, wherein at least a part of the aqueous phase obtained in said step (b) is fed to the hydrolysis of the lignocellulosic biomass.

19. The integrated process for the production of polyhydroxyalkanoates (PHAs) and bioethanol according to claim 1, wherein in said step (d) said microorganism capable to use both sugars with 5 carbon atoms (C5) and sugars with 6 carbon atoms (C6), is used at an initial cell concentration (dry weight) between 0.1 g/L and 2 g/L.

20. The integrated process for the production of polyhydroxyalkanoates (PHAs) and bioethanol according to claim 1, wherein in said step (d), the fermentation in said third fermentation device is carried out:
at a temperature between 20° C. and 40° C.; and/or
for a time between 1 day and 6 days; and/or
at a pH between 4 and 7; and/or
in one or more steps, in a batch mode, in a fed-batch mode, in continuous mode.
